(19) United States
(12) Patent Application Publication
(10) Pub. No.: US 2023/0363938 A1
(43) Pub. Date: Nov. 16, 2023

(54) POLYMERIC MANDIBULAR ADVANCEMENT DEVICES AND METHODS FOR MAKING AND USING THEM

(71) Applicant: Prosomnus Sleep Technologies Inc., Pleasanton, CA (US)

(72) Inventors: Sung KIM, Pleasanton, CA (US); David W. KUHNS, Pleasanton, CA (US); Leonard A. LIPTAK, Pleasanton, CA (US)

(21) Appl. No.: 18/028,453

(22) PCT Filed: Sep. 24, 2021
(86) PCT No.: PCT/US2021/052072

§ 371 Date: Mar. 24, 2023

Related U.S. Application Data
(60) Provisional application No. 63/082,945, filed on Sep. 24, 2020.

(51) Int. Cl. A61F 5/56; A61C 7/08; A61L 31/06

(52) U.S. Cl. CPC A61F 5/566; A61C 7/08; A61L 31/06; A61F 2005/563

(57) ABSTRACT

In alternative embodiments, provided are mandibular advancement devices (MADs) fabricated having polymeric materials to impart advantageous properties to the MADs. In alternative embodiments, MADs as provided herein comprise an upper splint and a lower splint, wherein the upper splint comprises one or more upper fins; the lower splints comprise one or more lower fins; wherein the upper and lower splints are made of a polymeric material (PM) having a Young's Modulus of between about 0.1 to about 10 GPa, and methods for making and using them. In alternative embodiments, provided are methods for treating bruxism or a sleep-related breathing disorder, wherein optionally the sleep-related breathing disorder is obstructive sleep apnea (OSA) or snoring, and/or a TMJ disorder comprising use of a MAD as provided herein.

### RELATED APPLICATIONS

This Patent Convention Treaty (PCT) International Application claims the benefit of priority under 35 U.S.C. § 119(e) of U.S. Provisional Application No. 63/082,945, filed Sep. 24, 2020. The aforementioned application is expressly incorporated herein by reference in its entirety and for all purposes. All publications, patents, patent applications cited herein are hereby expressly incorporated by reference for all purposes.

### TECHNICAL FIELD

The invention generally relates to dental devices, and in particular, is in the field of mandibular advancement devices (MADs) which are fabricated having polymeric materials to impart advantageous properties to the MADs.

### BACKGROUND

Mandibular Advancement Devices (MADs), also called mandibular splints or mandibular advancement splints, are prescription custom-made dental/medical devices worn in the mouth used to treat sleep-related breathing disorders including obstructive sleep apnea (OSA), snoring, and TMJ disorders. A survey study of patients found that many discontinued use because of discomfort or lack of efficacy, or because of the frequently reported side-effects, which include dry mouth, tooth pain, dental discomfort and jaw pain. For example, a MAD made by polymers currently used in the industry have the disadvantage that they cause the MAD to be too rigid, which itself can cause teeth to crack or break, or too bulky because of the need for additional strength. In either case, the MAD would be too uncomfortable for the patient to wear. Thus, there is a need for MADs manufactured with safe, comfortable and durable materials.

### SUMMARY

In alternative embodiments, provided are products of manufacture or dental devices such as mandibular advancement devices (MADs) comprising a polymeric material (PM) selected from the group consisting of a Class IV polymer, a Class V polymer, a Class VI polymer (as defined in Chapter 88 of the United States Pharmacopeia and National Formulary (USP-NF)), and combinations thereof, wherein the classes are defined in Chapter 88 of the United States Pharmacopeia and National Formulary (USP-NF).

In alternative embodiments, provided are products of manufacture or dental devices such as mandibular advancement devices (MADs) comprising: an upper splint and a lower splint, wherein the upper splint comprises one or more upper fins; the lower splints comprise one or more lower fins; wherein the upper and lower splints are made of, or comprise, or are manufactured from: at least one polymeric material (PM) having a Young's Modulus of between about 0.5 to about 3 GPa, or between about 0.1 to about 10 GPa, or about 0.5, 0.75, 1.0, 1.25, 1.5, 1.75, 2.0, 2.25, 2.5, 2.75 or 3 (gigapascals).

In alternative embodiments of products of manufacture or dental devices such as MADs as provided herein:
the MAD is milled as or in one piece;
the PM is or comprises, or is substantially comprised of:
   (a) a polymer comprising, or substantially comprising, an amorphous copolyester produced from or comprising: dimethyl terephthalate, 1,4-cyclohexanedimenthanol and 2,2,4,4,-tetremethyl-1,3-cyclobutanediol (the amorphous copolyester designated EVO), wherein optionally the upper and lower splints are manufactured from or milled from a polymer comprising at least about 60%, 70%, 80%, 90% or 95% EVO; or
   (b) a copolymer derived from, or comprising, two or more species of monomer comprising, or substantially comprising, an amorphous copolyester produced from or comprising: dimethyl terephthalate, 1,4-cyclohexanedimenthanol and 2,2,4,4,-tetremethyl-1,3-cyclobutanediol (the amorphous copolyester designated EVO),
   wherein optionally the upper and lower splints are manufactured from or milled from a polymer comprising at least about 40%, 50%, 60%, 70%, 80%, 90% or 95% EVO;
the PM is a copolymer derived from, or comprising, two or more species of monomer;
the PM is a combination of, or comprises, two or more copolymers;
the copolymers are introduced, or are fabricated or situated in the MAD, or in the body of the MAD: i) at random, ii) as block copolymers, or iii) a combination of i) and ii);
the PM is selected from the group consisting of a Class IV polymer, a Class V polymer, a Class VI polymer, and combinations thereof, wherein the classes are defined in Chapter 88 of the United States Pharmacopeia and National Formulary (USP-NF), and optionally the Class IV polymer, the Class V polymer and/or the Class VI polymer comprises a medical silicone or a polysiloxane, a medical grade polypropylene, polyurethane, a polycarbonate urethane, a polycarbonate-silicone urethane copolymer, a polyamine, a polyethylene terephthalate, a polycaprolactone, a polyvinyl chloride elastomer, a polyolefin homopolymeric and/or copolymeric elastomer, a urethane-based elastomer, a natural rubber or a synthetic rubber, or a mixture thereof, and the like;
more than about half of bonds, or between about 51% and 99% of bonds, between polymeric building blocks of the PM are ester bonds, carbonate bonds, or a combination thereof;
after bending one of the upper or lower splints for a time interval of TI at an angle of θ from an original position, the upper or lower splint returns to within an angle of co from the original position, wherein TI is between about 15 minutes to about 60 minutes, θ is between about 15° to about 90°, and ω is between about 1° to about 30°;
the TI is between about 20 minutes to about 45 minutes; or the TI is between about 30 minutes to about 35 minutes; or θ is between about 30° to about 60°; or θ is between about 40° to about 50°; or ω is between about 5° to about 20°; or ω is between about 8° to about 12°;
the curve of stress versus (vs.) strain for the PM has an area under the curve that is more than about twice, or more than about 4 times, or more than about 7 times, or more than about 12 times, greater than the analogous area under the curve for similar upper and lower splints made of polymethylmethacrylate (PMMA, also known as acrylic, perspex or plexiglass);
wherein the PM comprises one or more additional material(s) selected from the group consisting of reinforcing fillers, impact modifiers, tougheners, plasticizers, thermal stabilizers and combinations thereof;
wherein the PM comprises up to about 5%, or about 30%, or about 45%, or about 60%, or between about 1% and 75%, or 2% and 70%, of reinforcing fillers;
wherein the PM comprises up to about 1%, or about 5%, or about 10%, or about 20%, or between about 0.5% and 25%, of impact modifiers, tougheners or a combination thereof;
wherein the PM allows for prolonged or permanent exposure time, wherein these terms are defined in the United States Pharmacopeia and National Formulary (USP-NF);
wherein the PM is or comprises a polymer monolith, or the PM is or comprises a copolymer;
wherein the PM comprises less than about (or no more than about) 1%, or less than about 0.5%, or between about 0.25% and 2%, of leachables; and/or
wherein the area covered by the splints between the gingiva and the height of contour of the MAD is at least about 1.1 times, or about 1.2 times, or about 1.4 times, or about 1.7 times, or between about 1 and 2 times, greater than a similar area covered by an MAD made of polymethylmethacrylate (PMMA) for the same patient.

In alternative embodiments, provided are methods for making a mandibular advancement device (MAD) comprising an upper splint and a lower splint, wherein the upper splint comprises one or more upper fins, and the lower splints comprise one or more lower fins, the method comprising fabricating the upper and/or lower splints using at least one polymeric material (PM) having a Young's Modulus of between about 0.1 to about 10 GPa (gigapascals).

In alternative embodiments of methods as provided herein:
the polymeric material (PM) is selected by its ability to return to its original position when bent for a period of time, and after bending one of the upper or lower splints for a time interval of TI at an angle of θ from an original position, the upper or lower splint returns to within an angle of ω from the original position;
the time interval (TI) (for the MAD to return to its original position, or to substantially approximately return to its original position, when bent for a period of time) is between about 15 minutes to about 60 minutes, or TI is between about 20 minutes to about 45 minutes, or TI is between about 30 minutes to about 35 minutes;
θ is between about 15° to about 90°, or, θ is between about 30° to about 60°, or θ is between about 40° to about 50°, or ω is between about 1° to about 30°, or ω is between about 5° to about 20°, or ω is between about 8° to about 12°, or the curve of stress versus (vs.) strain for the polymeric material (PM) has an area under the curve that is more than about twice, or more than about 4 times, or more than about 7 times, or more than about 12 times, greater than the analogous area under the curve for similar upper and lower splints made of polymethylmethacrylate (PMMA, also known as acrylic, perspex or plexiglass);
the PM is a copolymer derived from, or comprising, two or more species of monomer, or, the PM is a combination of, or comprises, two or more copolymers;
the copolymers are introduced, or are fabricated or situated in the MAD, or mixed in the body of the MAD: i) at random, or ii) as block copolymers, or iii) a combination of i) and ii);
the PM is selected from the group consisting of a Class IV polymer, a Class V polymer, a Class VI polymer, and combinations thereof, wherein the classes are defined in Chapter 88 of the United States Pharmacopeia and National Formulary (USP-NF), and optionally the Class IV polymer, the Class V polymer and/or the Class VI polymer comprises a medical silicone or a polysiloxane, a medical grade polypropylene, polyurethane, a polycarbonate urethane, a polycarbonate-silicone urethane copolymer, a polyamine, a polyethylene terephthalate, a polycaprolactone, a polyvinyl chloride elastomer, a polyolefin homopolymeric and/or copolymeric elastomer, a urethane-based elastomer, a natural rubber or a synthetic rubber, or a mixture thereof;
more than about half of bonds, or between about 51% and 99% of bonds, between polymeric building blocks of the PM are ester bonds, carbonate bonds, or a combination thereof;
wherein after bending one of the upper or lower splints for a time interval of TI at an angle of θ from an original position, the upper or lower splint returns to within an angle of ω from the original position, wherein TI is between about 15 minutes to about 60 minutes, θ is between about 15° to about 90°, and ω is between about 1° to about 30°;
wherein the PM comprises one or more additional material(s) selected from the group consisting of reinforcing fillers, impact modifiers, tougheners, plasticizers, thermal stabilizers and combinations thereof;
wherein the PM comprises up to about 5%, or about 30%, or about 45%, or about 60%, or between about 1% and 75%, or 2% and 70%, of reinforcing fillers, or the PM comprises up to about 1%, or about 5%, or about 10%, or about 20%, or between about 0.5% and 25%, of impact modifiers, tougheners or a combination thereof, or the PM allows for prolonged or permanent exposure time, wherein these terms are defined in the United States Pharmacopeia and National Formulary (USP-NF);
the PM is or comprises a polymer monolith, or the PM is or comprises a copolymer and/or a copolymer derived from, or comprising, two or more species of monomer, or the PM comprises less than about (or no more than about) 1%, or less than about 0.5%, or between about 0.25% and 2%, of leachables;
the area covered by the splints between the gingiva and the height of contour of the MAD is at least about 1.1 times, or about 1.2 times, or about 1.4 times, or about 1.7 times, or between about 1 and 2 times, greater than a similar area covered by an MAD made of polymethyl-methacrylate (PMMA) for the same patient; and/or
the methods comprise calculating a needed spring constant (k) required for MAD and choosing a PM that exhibits that spring constant.

In alternative embodiments, provided are methods for treating bruxism or a sleep-related breathing disorder, wherein optionally the sleep-related breathing disorder is obstructive sleep apnea (OSA) or snoring, and/or a TMJ disorder, comprising administering to an individual in need thereof a mandibular advancement device (MAD) as provided herein.

In alternative embodiments, provided are mandibular advancement devices (MADs) for use in treating bruxism or a sleep-related breathing disorder, wherein optionally the sleep-related breathing disorder is obstructive sleep apnea (OSA) or snoring, and/or a TMJ disorder, wherein the MAD comprises a device as provided herein.

In alternative embodiments, provided are uses of a mandibular advancement device (MAD) for treating bruxism or a sleep-related breathing disorder, wherein optionally the sleep-related breathing disorder is obstructive sleep apnea (OSA) or snoring, and/or a TMJ disorder, wherein the MAD comprises a device as provided herein.

The details of one or more exemplary embodiments of the invention are set forth in the description below. Other features, objects, and advantages of the invention will be apparent from the description, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

FIG. 1 illustrates details of in vivo tests designated by the USP-NF to classify Plastics Class I through Class VI, as discussed in further detail, below.

FIG. 2 schematically illustrates a section of an exemplary MAD as provided herein fitting over an arch of teeth in situ, and illustrating the undercut of the device, which is partially responsible for retention of the device over the teeth, and because the MAD is manufactured with material having a Young's Modulus of between about 0.5 to about 3 GPa, for example, polymethylmethacrylate (PMMA) or a mix of PMMA and a polymer comprising, or substantially comprising, an amorphous copolyester produced from or comprising: dimethyl terephthalate, 1,4-cyclohexanedimenthanol and 2,2,4,4,-tetremethyl-1,3-cyclobutanediol (the amorphous copolyester designated EVO), or entirely of or substantially only EVO, the device's elasticity increases without compromising its ability to fit properly over the teeth, and with this increase in elasticity, the device (MAD) retention increases by over 50% while at the same time minimizing tooth movement and preventing the device (MAD) from coming loose (from the teeth). In FIG. 1 the PMMA undercut coverage is yellow (the upper part of the illustrated triangle), and the EVO undercut coverage includes both the yellow and green sections of the illustrated triangle); the flexible material in the undercut allows the device (MAD) to adaptable to a wider range of patients, including patients with challenging dentitions, for example, patients with short clinical crown teeth.

FIG. 3A graphically illustrates how EVO has 51% more flexibility than PMMA, noting that 51% less Young's modulus means 51% more flexibility, and with this material characteristic devices (MADs) as provided herein have better retention to the teeth in situ and be made by milling as one piece.

FIG. 3B graphically illustrates the Young's modulus, or stiffness, in GPa, where the lower the Young's modulus the less still the material.

FIG. 4 graphically illustrates how EVO retains its elasticity over time as compared to other polymers such as PMMA.

FIG. 5 graphically illustrates that the impact strength (in 1 ft-lbs/in, or 1 foot-pounds per inch) for EVO is almost ten times (10×) greater PMMA.

FIG. 6 illustrates that an exemplary device (MAD) as provided herein comprising (or manufactured using) EVO as the compositional polymer had a 3.7 impact strength, and passed a MIL-STD 810 drop test.

FIG. 7A-C illustrate design of the flexible portion of the device (the MAD) as provided herein that occupies the tooth's undercut (an undercut is that portion of a tooth that lies between its height of contour and the gingiva, only if that portion is of less circumference than the height of contour), in other words, these illustrations aid in determining the dimensions and contour of how much of the device should occupy a tooth's undercut:

FIG. 7A: schematically illustrates the contour of a tooth and it's undercut dimensions;

FIG. 7B schematically illustrates the contour of a tooth and in the shaded (or blue) area the amount of space to be occupied by the device (MAD) in the undercut when the device is fabricated with PMMA; and,

FIG. 7C schematically illustrates the contour of a tooth and in the shaded (or yellow) area the amount of space to be occupied by the device (MAD) in the undercut when the device is fabricated with EVO.

Like reference symbols in the various drawings indicate like elements.

### DETAILED DESCRIPTION

Disclosed herein are various polymeric materials (PMs) that are especially suitable for the manufacture of a mandibular advancement device (MAD). Also disclosed are MADs manufactured from the disclosed PMs. In alternative embodiments, provided are mandibular advancement device (MAD) comprising an upper splint and a lower splint, wherein the upper splint comprises one or more upper fins; the lower splints comprise one or more lower fins; wherein the upper and lower splints are made of a polymeric material (PM) having a Young's Modulus of between about 0.1 to about 10 GPa (gigapascals).

Mandibular Advancement Devices (MADs) comprising polymeric materials (PMs) as provided herein can be adapted for use with, used for manufacturing, or designed as MADs as disclosed in U.S. Pat. No. 9,820,882; or MADs as disclosed in U.S. Pat. No. 9,808,327, describing automated methods for manufacturing MADS, and MADs as disclosed in the US Patent Application Publication 2018/0024530, describing a computer aided design matrix for MAD manufacture. The entire disclosure of these references, including all drawings therein, are incorporated by reference in their entirety herein, especially sections of the references that discuss the shape, function, and manufacture of the MADs.

In one aspect, the MADs as provided herein are manufactured with or from the presently disclosed PMs, wherein the MADs comprise an upper splint and a lower splint, where the upper splint comprises one or more upper fins; and the lower splints comprise one or more lower fins. In some embodiments, the fins of the MADs comprise a sleeve, as disclosed in the US Patent Application Publication 2019/0105191, the entire disclosure of which, including all the drawings, is incorporated by reference herein, especially sections that discuss the shape, function, and manufacture of the disclosed fin sleeves.

U.S. Pat. Nos. 9,820,882, 9,808,327, US Patent Application Publication 2018/0024530, and US Patent Application Publication 2019/0105191 are collectively referred to as the “above-incorporated references.”

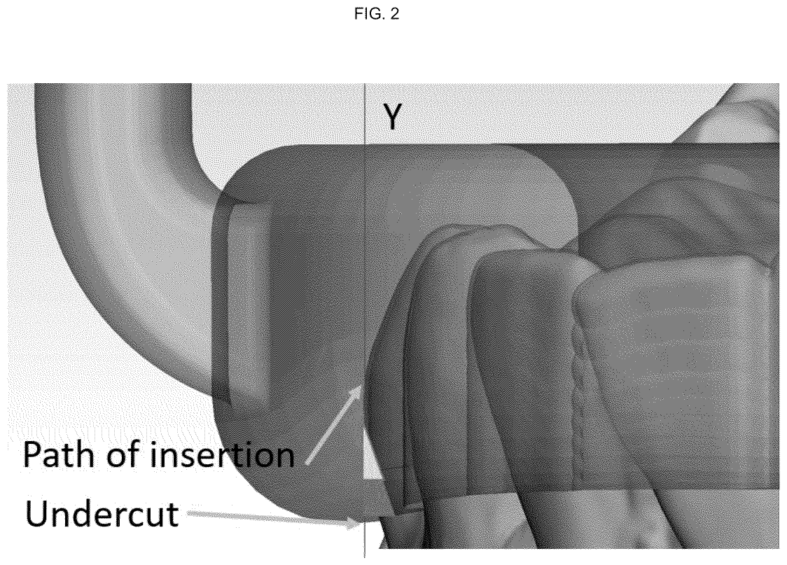

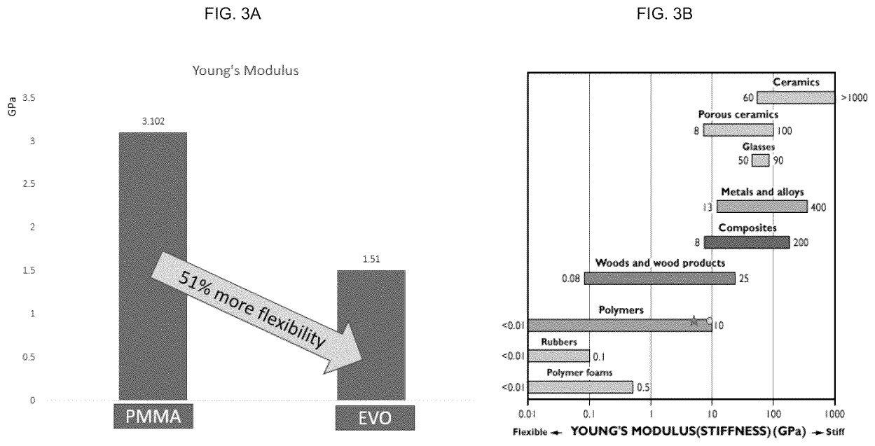

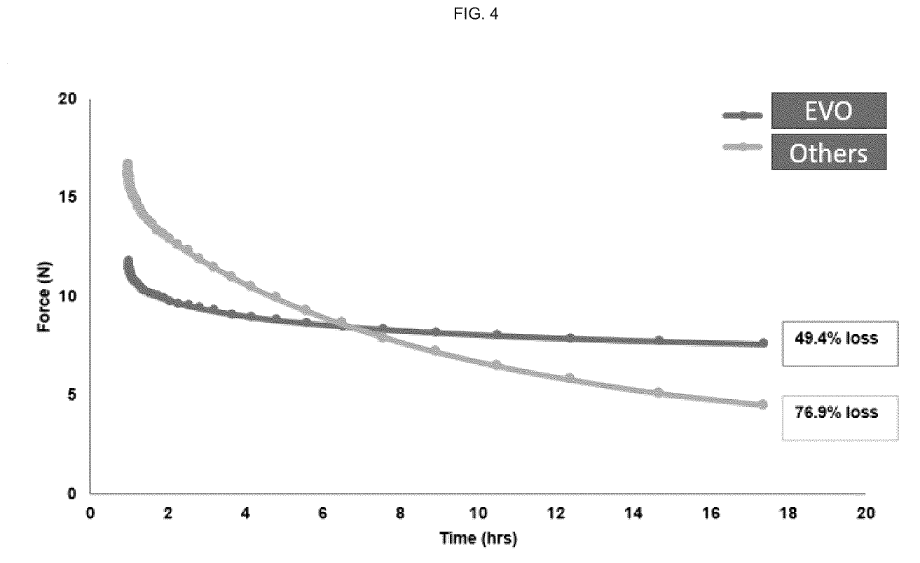

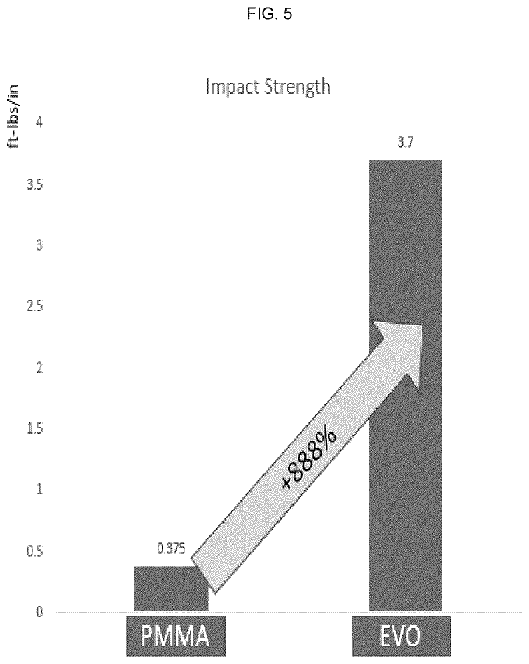

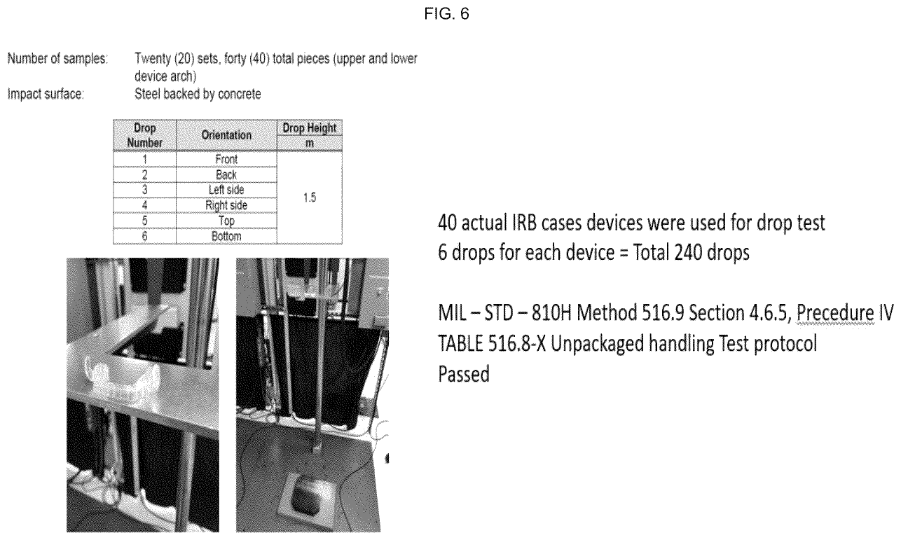

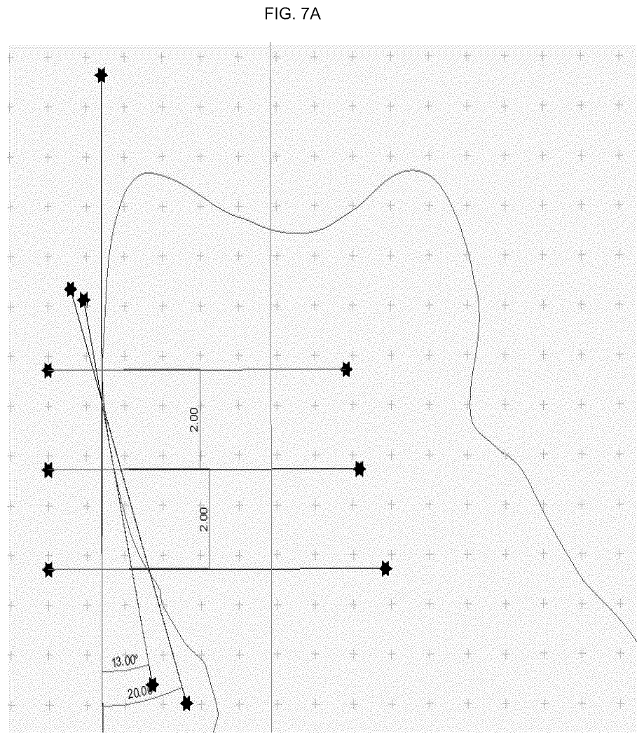

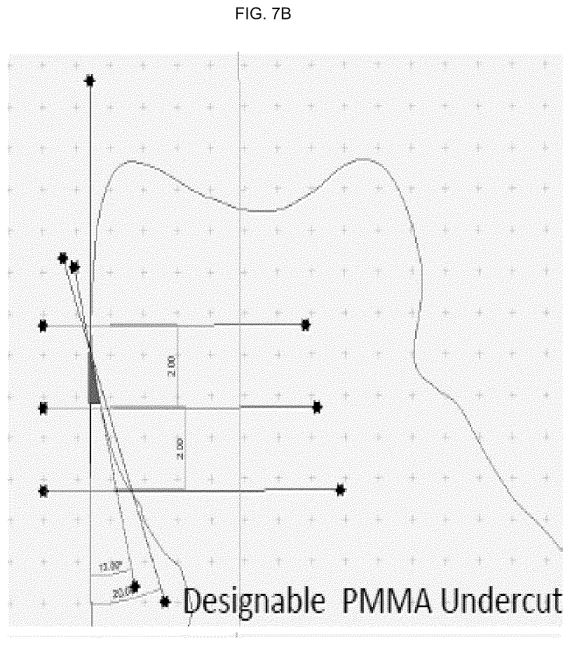

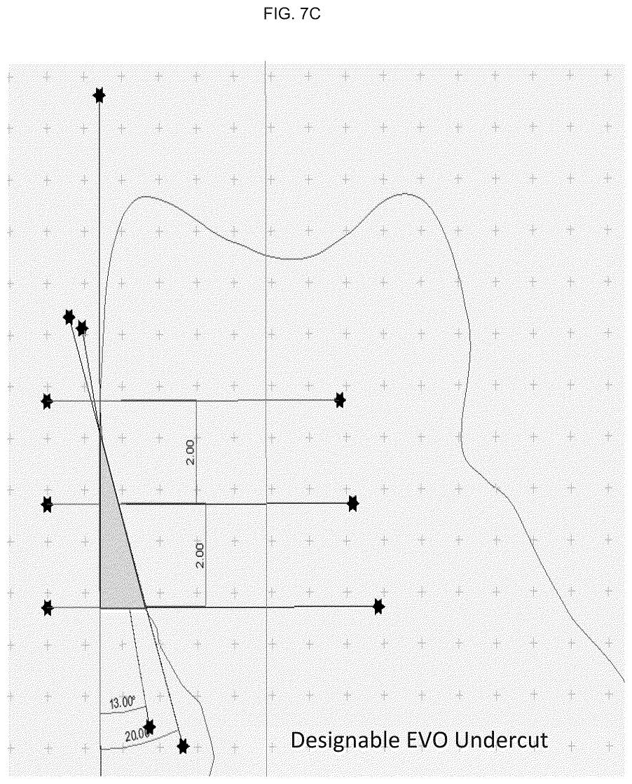

In alternative embodiments, devices (MADs) as provided herein are designed and fabricated to have sections of the device occupy a tooth's undercut, as illustrated in FIG. 2, and FIG. 7A-C. In alternative embodiments, the flexible material of a device as provided herein in the undercut allows the device (MAD) to be adaptable to a wider range of patients, including patients with challenging dentitions, for example, patients with short clinical crown teeth. In alternative embodiments, undercuts are designed as described in U.S. Pat. Nos. 10,925,690; 10,123,852; and, 9,364,296; and U.S. patent application publication nos. US20090248184A1 and US20060263739A1.

One of the criteria used in selecting a PM for the manufacture of the MADs is the flexibility and stiffness of the PM. A stiff PM is desirable because it does not deform under the pressure exerted by the maxilla and the mandible during the advancement phase. With a stiff PM, a 1 mm advancement of the mandible translates to a true 1 mm advancement. With a flexible PM, it is unknown what the true advancement would be because the PM itself flexes without moving the mandible.

On the other hand, a stiff PM may cause enough pain and discomfort for the patient to discontinue wear. In addition, because MADs are held in place by friction, when the flexible PM deforms slightly it can take on the contours of the teeth, providing a greater surface-to-surface contact, which results in greater friction and a better fit of the MAD. Stiff polymeric MADs are incapable of such deformation and consequently may fall out of the mouth much more easily than the flexible polymeric MADs.

The stiffness of PMs is defined using Young's modulus. Young's modulus is calculated using the following formula:

$$E = \frac{σ}{ε}$$

where E is Young's modulus, σ is the uniaxial stress, or uniaxial force, per unit surface, and ε is the strain, or the dimensionless proportional deformation (change in length divided by original length).

After experimenting with multiple PMs, the present inventors have discovered that in some embodiments, the PM from which the presently disclosed MADs are manufactured, is a PM having a Young's modulus of between about 0.1 to about 10 GPa (gigapascal), or in other embodiments, between about 0.5 to about 5 GPa.

By “about” a certain value it is meant that the stated value comprises the range of values within ±25%, ±20%, ±10%, or ±5% of the stated value. Thus, by way of example only, if a distance is given as “about 5 mm,” the range of distances between 3.75 mm (5-25%) to 6.25 mm (5+25%) is envisioned.

In some embodiments, the PM is a copolymer derived from two or more species of monomer. In certain embodiments, the PM is a combination of two or more copolymers. In some embodiments the copolymers are introduced at random whereas in other embodiments, the copolymers are introduced as block copolymers. In still other embodiments, a combination of copolymer random distribution and block copolymers are used.

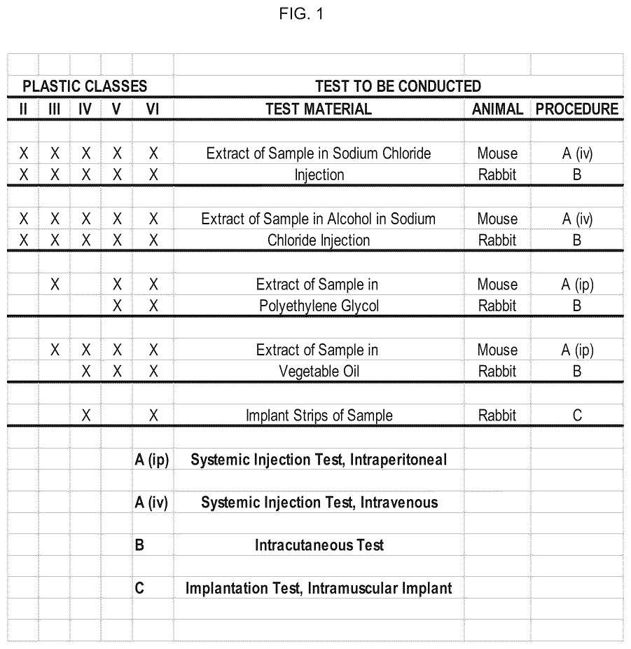

In alternative embodiments, the PM is a plastic is selected from the group consisting of a Class IV polymer, a Class V polymer, a Class VI polymer, and combinations thereof, as the classes are defined in Chapter 88 of the United States Pharmacopeia and National Formulary (USP-NF), which definitions are incorporated herein by reference. Chapter 88 provides for the in vivo testing of elastomers, plastics, polymeric materials and their extracts. This in vivo testing consists of three (3) tests: Systemic, Intracutaneous, and Implantation. The materials and their extracts are then classified according to the test results as meeting USP-NF Plastics Class I through Class VI; and a summary of these tests are set forth in FIG. 1. The above USP-NF Plastics Class Testing of elastomers, plastics, polymeric materials and their extracts is performed in contact with animals. In alternative embodiments, PMs used in MADs as provided herein are also classified as GRAS (Generally Recognized as Safe) material.

In alternative embodiments, Class IV, V and VI PMs used in MADs as provided herein comprise a medical silicone or a polysiloxane, a medical grade polypropylene, polyurethane, a polycarbonate urethane, a polycarbonate-silicone urethane copolymer, a polyamine, a polyethylene terephthalate, a polycaprolactone, a polyvinyl chloride elastomer, a polyolefin homopolymeric and/or copolymeric elastomer, a urethane-based elastomer, a natural rubber or a synthetic rubber, or a mixture thereof, and can be bisphenol A (BPA)-, lead- and heavy metal free.

In some embodiments, about more than half, or between about 51% and 99%, of bonds between the polymeric building blocks of the PM are ester bonds.

In other embodiments, about more than half, or between about 51% and 99%, of bonds between the polymeric building blocks of the PM are carbonate bonds.

In other embodiments, more than half of bonds between the polymeric building blocks of the PM, or between about 51% and 99%, are either ester bonds or carbonate bonds or a mixture of the two.

In some embodiments, the polymeric material (PM) has the ability to return to its original position when bent for a period of time. In some embodiments, after bending one of the upper or lower splints for a time interval of TI at an angle of θ from an original position, the upper or lower splint returns to within an angle of ω from the original position.

In some embodiments, the time interval (TI) (for the MAD to return to its original position, or to substantially approximately return to its original position, when bent for a period of time) is between about 15 minutes to about 60 minutes, while in other embodiments, TI is between about 20 minutes to about 45 minutes. In still other embodiments, TI is between about 30 minutes to about 35 minutes. In some embodiments, θ is between about 15° to about 90°, while in other embodiments, θ is between about 30° to about 60°. In still other embodiments, θ is between about 40° to about 50°. In some embodiments, ω is between about 1° to about 30°, while in other embodiments, ω is between about 5° to about 20°. In still other embodiments, ω is between about 8° to about 12°.

In some embodiments, the curve of stress versus (vs.) strain for the polymeric material (PM) has an area under the curve that is more than about twice, or more than about 4 times, or more than about 7 times, or more than about 12 times, greater than the analogous area under the curve for similar upper and lower splints made of PMMA.

In some embodiments, the polymeric material (PM) comprises one or more additional material(s) selected from the group consisting of reinforcing fillers, impact modifiers or tougheners, plasticizers, thermal stabilizers and combinations thereof. These terms carry their art-accepted definitions.

In some embodiments, the polymeric material (PM) comprises up to about 5%, or about 30%, or about 45%, or about 60%, or between about 15 and 65%, of reinforcing fillers. In other embodiments, the PM comprises up to about 1%, or about 5%, or about 10%, or about 20%, or between about 0.5% and 30%, of impact modifiers or tougheners. In still other embodiments, the impact modifier or toughener improves ambient temperature impact strength. By “ambient temperature” it is meant the temperature of the ambient where the MAD is found. This includes room temperature (RT), anywhere between about 10° C. to about 30° C., and body temperature of between about 35° C. to about 39° C. While these temperature ranges are discussed in terms of the MADs impact resistance, it is noted that a PM as used herein, or a MAD as provided herein, by itself and without the force of an impact, would survive without any damage much higher temperatures, such as those encountered in an autoclave or other sterilization or disinfection environments.

In some embodiments, the PM allows for prolonged or permanent exposure time, as these terms are defined in the United States Pharmacopeia and National Formulary (USP-NF), which definitions are incorporated herein by reference.

In some embodiments, the PM is a polymer monolith, while in other embodiments, as discussed above, the PM is a copolymer.

In some embodiments, the PM comprises less than about 1% of leachables, in other words, the PM can comprise compounds and/or other material(s) that can leach out from the polymer under physiological conditions of the mouth. In alternative embodiments, the leachables are non-toxic.

The height of contour is considered when designing MADs as provided herein. “Height of contour,” as that term is fully defined in one or more of the above-incorporated references, is a point visible to the eye on the buccal side of the molar, where the tooth is widest along the xz plane (for example, where the tooth has the widest radius along the either the x axis (left-right direction) or the z axis (anterior-posterior direction)).

The incorporation of the height of contour in the design of MADs as provided herein is significant because an MAD must pass the height of contour into the area between the gingiva and the height of contour in order to stay affixed in the mouth. If the height of the MAD is lower than that of the height of contour, then the MAD falls out easily. However, when the MAD height is higher than the height of contour, and the MAD curves slightly inward to capture the width of the tooth, a relatively stable friction lock is created that holds the MAD in place in the mouth until the patient intentionally removes the MAD. Accordingly, the more the MAD height encroaches into the space between the gingiva and the height of contour, the better the MAD stays in the mouth.

However, if the PM that forms the MAD is too stiff, then removing the MAD will be difficult, painful, and could even cause the tooth to become loose or fall out. Thus, how much higher than the height of contour should the height of the MAD be will depend heavily on the type of material used for the manufacture of the MAD. Therefore, a PM is needed to provide sufficient grip, without exerting too much grip, and PMs used to manufacture MADs as provided herein provide sufficient grip, without exerting too much grip. In other words, PMs are used to manufacture MADs as provided herein are of sufficient stiffness such that the MAD can conform to the contour of the tooth and grab the tooth at the height of contour, but be malleable enough for the patient to remove it without exerting too much force or experiencing pain.

Polymethylmethacrylate (PMMA) is a common polymer used in the manufacture of MADs. However, PMMA may be too stiff and cause patient discomfort. Further, because of its stiffness and discomfort, the height of a PMMA splint can by only a certain distance above the height of contour. If additional grip is required, it cannot be achieved by increasing the height of contour, as patient discomfort reduces patient compliance dramatically.

Accordingly, an exemplary splint or MAD made for a patient using PMs as disclosed herein was compared with a splint made of polymethylmethacrylate (PMMA) for the same patient. Both splints covered an area between the gingiva and the height of contour. The area covered by the presently disclosed MADs was at least about 1.1 times, or about 1.2 times, or about 1.4 times, or about 1.7 times greater than the area covered by the PMMA splint, without sacrificing patient comfort or ease of removal. The results showed that the presently disclosed PMs provide a much better grip at the same, or higher, level of comfort.

In alternative embodiments, MADs as provided herein are used to treat a variety of disorders including bruxism, temporomandibular joint (TMJ)-related disorders, and/or orthodontic needs, in conjunction with their ability and function to advance the mandible. Bruxism in patients has a range of motion that is difficult to predict but is often reduced upon alleviation of the cause (for example, sleep apnea or TMJ related disorders).

In alternative embodiments, one aspect of the treatment for these disorder comprises the design and use of MADs as provided herein which can force the patient's dentition from a position dictated by the musculoskeletal architecture of the patient's face to a position dictated by a healthcare professional (HPC) (“HPC” is fully defined in one or more of the above-incorporated references.) The position as dictated by the HPC is aimed to provide treatment, such as better airway management during sleep, reduction of force on teeth by other teeth, or the proper alignment of the teeth.

In patients who suffer other oral disorders, such as bruxism or TMJ related disorders, in addition to sleep apnea, the patient's teeth rub or push against each other with such force that cause damage to the teeth, such as wearing away, cracking, or breaking of the teeth. Any force that can break a tooth is also strong enough to break an MAD working against that force. Accordingly, in alternative embodiments MADs as provided herein are made of PMs that are strong enough to withstand the pressure exerted by the teeth on the teeth. A MAD made by polymers currently used in the industry have the disadvantage that they cause the MAD to be too rigid, which itself can cause teeth to crack or break, or too bulky because of the need for additional strength. In either case, the MAD would be too uncomfortable for the patient to wear.

In alternative embodiments, the MADs made from the PMs disclosed herein treat the or an underlying cause of bruxism or a TMJ-related disorder, while at the same time they are comfortable for the patient to wear during the disease alleviation process. By using a flexible PM in a MAD as provided herein, as described herein, in the design of the exemplary oral appliances, the PM acts as a spring that changes its conformation slightly upon the exertion of a force but returns to its original conformation when the force is removed. Just like a spring, a PM having the attributes disclosed herein is designed to absorb the pressure applied to it.

An HCP can determine the extent of the patient's jaw movement during a bruxism episode. From this information, a person having ordinary skill in the art (POSITA) can determine the extent of forces that are applied to the jaws and the dentition during the episode. The POSITA can then incorporate this information into the design of an exemplary MAD appliance as provided herein and the choice of a PM providing the greatest efficacy.

In one embodiment, the POSITA uses Hooke's Law to determine the extent of play, usually between about 0 mm to about 10 mm, that needs to be incorporated into the device. Deformation of this size are within the parameters of the disclosed PMs where Hooke's Law is applicable.

Hooke's law is a law of physics that states that the force (F) needed to extend or compress a spring by some distance (x) scales linearly with respect to that distance—that is, F=kx, where k is a constant factor characteristic of the spring (i.e., its stiffness), and x is small compared to the total possible deformation of the spring. While Hooke's law was originally defined with respect to springs, a POSITA recognizes that it applies to all elastic bodies that behave Hookean, for example, the PMs disclosed herein. Thus, knowing the amount of force (F) needed by knowing the physiology of the mouth, and knowing the extent of play required (x) from the HCP's prescription, the POSITA can calculate the spring constant (k) required for the particular device and choose a PM that exhibits that spring constant. Thus, in some embodiments, the choice of a PM ultimately depends on the findings of the HCP and the HCP's prescription for extent of play in the desired exemplary oral appliance.

In alternative embodiments, provided are MADs comprising PMs that are flexible, and in conjunction with the design of the MAD's posts or fins, provide a wider range of lateral motion due to the flex of the material, but also support the comfort enabled by a low profile device as the symptoms of bruxism alleviate.

### Products of Manufacture and Kits

Provided are products of manufacture fabricated as MADs as provided herein, and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

A number of embodiments of the invention have been described. Nevertheless, it can be understood that various modifications may be made without departing from the spirit and scope of the invention. Accordingly, other embodiments are within the scope of the following claims.

1: A mandibular advancement device (MAD) comprising an upper splint and a lower splint, wherein:
the upper splint comprises one or more upper fins;
the lower splints comprise one or more lower fins;
wherein the upper and lower splints are made of, or comprise, or manufactured from, at least one polymeric material (PM) having a Young's Modulus of between about 0.5 to about 3 GPa (gigapascals), or about or about 0.5, 0.75, 1.0, 1.25, 1.5, 1.75, 2.0, 2.25, 2.5, 2.75 or 3 GPa (gigapascals).

2: The MAD of claim 1, wherein the PM is or comprises:
(a) a polymer comprising, or substantially comprising, an amorphous copolyester produced from or comprising: dimethyl terephthalate, 1,4-cyclohexanedimenthanol and 2,2,4,4,-tetremethyl-1,3-cyclobutanediol (the amorphous copolyester designated EVO),
wherein optionally the upper and lower splints are manufactured from or milled from a polymer comprising at least about 60%, 70%, 80%, 90% or 95% EVO; or
(b) a copolymer derived from, or comprising, two or more species of monomer comprising, or substantially comprising, an amorphous copolyester produced from or comprising: dimethyl terephthalate, 1,4-cyclohexanedimenthanol and 2,2,4,4,-tetremethyl-1,3-cyclobutanediol (the amorphous copolyester designated EVO),
wherein optionally the upper and lower splints are manufactured from or milled from a polymer comprising at least about 40%, 50%, 60%, 70%, 80%, 90% or 95% EVO.

3: The MAD of claim 1, wherein the PM is a combination of, or comprises, two or more copolymers.

4: The MAD of claim 3, wherein the copolymers are introduced, or are fabricated or situated in the MAD, or in the body of the MAD:
i) at random, or
ii) as block copolymers, or
iii) a combination of i) and ii).

5: The MAD of claim 1, wherein the PM is selected from the group consisting of a Class IV polymer, a Class V polymer, a Class VI polymer, and combinations thereof, wherein the classes are defined in Chapter 88 of the United States Pharmacopeia and National Formulary (USP-NF),
and optionally the Class IV polymer, the Class V polymer and/or the Class VI polymer comprises a medical silicone or a polysiloxane, a medical grade polypropylene, polyurethane, a polycarbonate urethane, a polycarbonate-silicone urethane copolymer, a polyamine, a polyethylene terephthalate, a polycaprolactone, a polyvinyl chloride elastomer, a polyolefin homopolymeric and/or copolymeric elastomer, a urethane-based elastomer, a natural rubber or a synthetic rubber, or a mixture thereof, and the like.

6: The MAD of claim 1, wherein more than about half of bonds, or between about 51% and 99% of bonds, between polymeric building blocks of the PM are ester bonds, carbonate bonds, or a combination thereof.

7: The MAD of claim 1, wherein after bending one of the upper or lower splints for a time interval of TI at an angle of θ from an original position, the upper or lower splint returns to within an angle of ω from the original position, wherein TI is between about 15 minutes to about 60 minutes, θ is between about 15° to about 90°, and ω is between about 1° to about 30°.

8: The MAD of claim 7, wherein TI is between about 20 minutes to about 45 minutes.

9. (canceled)

10: The MAD of claim 7, wherein θ is between about 30° to about 60°, or θ is between about 40° to about 50°.

11. (canceled)

12: The MAD of claim 7, wherein ω is between about 5° to about 20°, or ω is between about 8° to about 12°.

13. (canceled)

14: The MAD of claim 1, wherein the curve of stress versus (vs.) strain for the PM has an area under the curve that is more than about twice, or more than about 4 times, or more than about 7 times, or more than about 12 times, greater than the analogous area under the curve for similar upper and lower splints made of polymethylmethacrylate (PMMA).

15: The MAD of claim 1, wherein the PM comprises one or more additional material(s) selected from the group consisting of reinforcing fillers, impact modifiers, tougheners, plasticizers, thermal stabilizers and combinations thereof.

16: The MAD of claim 15, wherein the PM comprises up to about 5%, or about 30%, or about 45%, or about 60%, or between about 1% and 75%, or 2% and 70%, of reinforcing fillers.

17: The MAD of claim 15, wherein the PM comprises up to about 1%, or about 5%, or about 10%, or about 20%, or between about 0.5% and 25%, of impact modifiers, tougheners or a combination thereof.

18: The MAD of claim 1, wherein the PM allows for prolonged or permanent exposure time, wherein these terms are defined in the United States Pharmacopeia and National Formulary (USP-NF).

19: The MAD of claim 1, wherein the PM is or comprises a polymer monolith, or the PM is or comprises a copolymer; or the PM is a copolymer derived from, or comprising, two or more species of monomer.

20. (canceled)

21: The MAD of claim 1, wherein the PM comprises less than about or no more than about 1%, or less than about 0.5%, or between about 0.25% and 2%, of leachables.

22: The MAD of claim 1, wherein the area covered by the splints between the gingiva and the height of contour of the MAD is at least about 1.1 times, or about 1.2 times, or about 1.4 times, or about 1.7 times, or between about 1 and 2 times, greater than a similar area covered by an MAD made of polymethylmethacrylate (PMMA) for the same patient.

23: A method for making a mandibular advancement device (MAD) comprising an upper splint and a lower splint, wherein the upper splint comprises one or more upper fins, and the lower splints comprise one or more lower fins,
the method comprising fabricating the upper and/or lower splints using at least one polymeric material (PM) having a Young's Modulus of between about 0.1 to about 10 GPa (gigapascals).

24: The method of claim 23, wherein:
(a) the polymeric material (PM) is selected by its ability to return to its original position when bent for a period of time, and after bending one of the upper or lower splints for a time interval of TI at an angle of θ from an original position, the upper or lower splint returns to within an angle of ω from the original position;
(b) the time interval (TI) for the MAD to return to its original position, or to substantially approximately return to its original position, when bent for a period of time, is between about 15 minutes to about 60 minutes, or TI is between about 20 minutes to about 45 minutes, or TI is between about 30 minutes to about 35 minutes;
(c) θ is between about 15° to about 90°, or, θ is between about 30° to about 60°, or θ is between about 40° to about 50°;
(d) ω is between about 1° to about 30°, or ω is between about 5° to about 20°, or ω is between about 8° to about 12°;
(e) the curve of stress versus strain for the polymeric material (PM) has an area under the curve that is more than about twice, or more than about 4 times, or more than about 7 times, or more than about 12 times, greater than the analogous area under the curve for similar upper and lower splints made of polymethyl-methacrylate (PMMA);
(f) the PM is a copolymer derived from, or comprising, two or more species of monomer, or, the PM is a combination of, or comprises, two or more copolymers;
(g) copolymers are introduced, or are fabricated or situated in the MAD, or mixed in the body of the MAD: i) at random, ii) as block copolymers, or iii) a combination of i) and ii);
(h) the PM is selected from the group consisting of a Class IV polymer, a Class V polymer, a Class VI polymer, and combinations thereof, wherein the classes are defined in Chapter 88 of the United States Pharmacopeia and National Formulary (USP-NF), and optionally the Class IV polymer, the Class V polymer and/or the Class VI polymer comprises a medical silicone or a polysiloxane, a medical grade polypropylene, polyurethane, a polycarbonate urethane, a polycarbonate-silicone urethane copolymer, a polyamine, a polyethylene terephthalate, a polycaprolactone, a polyvinyl chloride elastomer, a polyolefin homopolymeric and/or copolymeric elastomer, a urethane-based elastomer, a natural rubber or a synthetic rubber, or a mixture thereof;
(i) more than about half of bonds, or between about 51% and 99% of bonds, between polymeric building blocks of the PM are ester bonds, carbonate bonds, or a combination thereof;
(k) after bending one of the upper or lower splints for a time interval of TI at an angle of θ from an original position, the upper or lower splint returns to within an angle of ω from the original position, wherein TI is between about 15 minutes to about 60 minutes, θ is between about 15° to about 90°, and ω is between about 1° to about 30°;
(l) the PM comprises one or more additional material(s) selected from the group consisting of reinforcing fillers, impact modifiers, tougheners, plasticizers, thermal stabilizers and combinations thereof;
(m) the PM comprises up to about 5%, or about 30%, or about 45%, or about 60%, or between about 1% and 75%, or 2% and 70%, of reinforcing fillers;
(n) the PM comprises up to about 1%, or about 5%, or about 10%, or about 20%, or between about 0.5% and 25%, of impact modifiers, tougheners or a combination thereof;
(o) the PM allows for prolonged or permanent exposure time, wherein these terms are defined in the United States Pharmacopeia and National Formulary (USP-NF);
(p) the PM is or comprises a polymer monolith, or the PM is or comprises a copolymer and/or a copolymer derived from, or comprising, two or more species of monomer;
(q) the PM comprises less than about (or no more than about) 1%, or less than about 0.5%, or between about 0.25% and 2%, of leachables;
(r) the area covered by the splints between the gingiva and the height of contour of the MAD is at least about 1.1 times, or about 1.2 times, or about 1.4 times, or about 1.7 times, or between about 1 and 2 times, greater than a similar area covered by an MAD made of polymethyl-methacrylate (PMMA) for the same patient; and/or
(s) the method further comprises calculating a needed spring constant (k) required for MAD and choosing a PM that exhibits that spring constant.

24-41. (canceled)

42: A method for treating bruxism or a sleep-related breathing disorder,
wherein optionally the sleep-related breathing disorder is obstructive sleep apnea (OSA) or snoring, and/or a TMJ disorder,
comprising administering to an individual in need thereof a mandibular advancement device (MAD) as set forth in claim 1.

43-44. (canceled)
